SEQUENCE LISTING (24 sequences)

SEQ ID NO: 1 - DNA, 20 bp, Artificial Sequence
  GAPDH forward primer
gtcagtggtggacctgacct

SEQ ID NO: 2 - DNA, 20 bp, Artificial Sequence
  GAPDH reverse primer
caccaccctgttgctgtagc

SEQ ID NO: 3 - DNA, 20 bp, Artificial Sequence
  GFAP forward primer
caacctgcagattcgagaaa

SEQ ID NO: 4 - DNA, 20 bp, Artificial Sequence
  GFAP reverse primer
gtcctgcctcacatcacatc

SEQ ID NO: 5 - DNA, 21 bp, Artificial Sequence
  Nestin forward primer
cacctgtgccagcctttctta

SEQ ID NO: 6 - DNA, 20 bp, Artificial Sequence
  Nestin reverse primer
tttcctcccaccctgtgtct

SEQ ID NO: 7 - DNA, 20 bp, Artificial Sequence
  Sox2 forward primer
agtctccaagcgacgaaaaa

SEQ ID NO: 8 - DNA, 20 bp, Artificial Sequence
  Sox2 reverse primer
gcaagaagcctctccttgaa

SEQ ID NO: 9 - DNA, 21 bp, Artificial Sequence
  Pax6 forward primer
aggtattacgagactggctcc

SEQ ID NO: 10 - DNA, 22 bp, Artificial Sequence
  Pax6 reverse primer
tcccgcttatactgggctattt

SEQ ID NO: 11 - DNA, 21 bp, Artificial Sequence
  Vimentin forward primer
agaactttgccgttgaagctg

SEQ ID NO: 12 - DNA, 23 bp, Artificial Sequence
  Vimentin reverse primer
ccagagggagtgaatccagatta

SEQ ID NO: 13 - DNA, 19 bp, Artificial Sequence
  sox10 forward primer
cggaccagtacccgcacct

SEQ ID NO: 14 - DNA, 21 bp, Artificial Sequence
  sox10 reverse primer
ggcgcttgtcactttcgttca

SEQ ID NO: 15 - DNA, 20 bp, Artificial Sequence
  S100beta forward primer
ggagacggcgaatgtgactt

SEQ ID NO: 16 - DNA, 23 bp, Artificial Sequence
  S100beta reverse primer
gaactcgtggcaggcagtagtaa

SEQ ID NO: 17 - DNA, 22 bp, Artificial Sequence
  PDGFR alpha forward primer
tacacttgctattacaaccaca

SEQ ID NO: 18 - DNA, 20 bp, Artificial Sequence
  PDGFR alpha reverse primer
atcctccacgatgactaaat

SEQ ID NO: 19 - DNA, 22 bp, Artificial Sequence
  SLC1A3 forward primer
ggttgctgcaagcactcatcac

SEQ ID NO: 20 - DNA, 22 bp, Artificial Sequence
  SLC1A3 reverse primer
cacgccattgttctcttccagg

SEQ ID NO: 21 - DNA, 22 bp, Artificial Sequence
  SLC1A2 forward primer
tgccaacagaggacatcagcct

SEQ ID NO: 22 - DNA, 22 bp, Artificial Sequence
  SLC1A2 reverse primer
cagctcagacttggagaggtga

SEQ ID NO: 23 - DNA, 21 bp, Artificial Sequence
  PAPP A forward primer
cagaatgcactgttacctgga

SEQ ID NO: 24 - DNA, 21 bp, Artificial Sequence
  PAPP A reverse primer
gctgatcccaattctctttca

(19) United States
(12) Patent Application Publication
(10) Pub. No.: US 2022/0339198 A1
(43) Pub. Date: Oct. 27, 2022

(54) PHARMACEUTICAL COMPOSITION FOR ALZHEIMER'S TREATMENT CONTAINING AS ACTIVE INGREDIENT LATE PASSAGE HUMAN MESENCHYMAL STEM CELLS INDUCED TO DIFFERENTIATE INTO GLIA-LIKE CELLS

(71) Applicants: SEOUL NATIONAL UNIVERSITY R&DB FOUNDATION, Seoul (KR); IUCF-HYU (INDUSTRY-UNIVERSITY COOPERATION FOUNDATION HANYANG UNIVERSITY), Seoul (KR)

(72) Inventors: Mi-Sook Chang, Seoul (KR); Seong-Ho Koh, Seoul (KR)

(73) Assignees: SEOUL NATIONAL UNIVERSITY R&DB FOUNDATION, Seoul (KR); IUCF-HYU (INDUSTRY-UNIVERSITY COOPERATION FOUNDATION HANYANG UNIVERSITY), Seoul (KR)

(21) Appl. No.: 17/615,575

(22) PCT Filed: Aug. 24, 2020
(86) PCT No.: PCT/KR2020/011248

§ 371 Date: Nov. 30, 2021

(30) Foreign Application Priority Data
Aug. 22, 2019 (KR) 10-2019-0103217

(51) Int. Cl. A61K 35/30; A61P 25/28; A61K 35/28

(52) U.S. Cl. CPC A61K 35/30; A61P 25/28; A61K 35/28

(57) ABSTRACT

The present invention relates to a pharmaceutical composition for Alzheimer's treatment containing as an active ingredient of late-stage human mesenchymal stem cells induced into glia-like cells (ghMSCs). When the glia-like cells differentiated from the late-stage human mesenchymal stem cells were co-cultured with neural stem cells having toxicity induced by amyloid beta, effects of increasing the reduced viability and proliferative potential of the neural stem cells and reducing the increased cytotoxicity of the neural stem cells were verified. In addition, the expression of inflammasomes is reduced, and effects of improving long-term memory with respect to spatial perception ability and enhancing spatial cognitive ability in Alzheimer-induced mouse models were verified. Therefore, the pharmaceutical composition of the present invention can be advantageously used in Alzheimer's treatment.

### BACKGROUND OF THE INVENTION

### 1. Field of the Invention

The present invention relates to a pharmaceutical composition comprising late-stage human mesenchymal stem cells induced into glia-like cells (ghMSCs) as an active ingredient for Alzheimer's treatment.

### 2. Description of the Related Art

Alzheimer's disease (AD) is a typical neurodegenerative disease associated with aging. Alzheimer's disease gradually progresses by affecting different areas of the brain starting from the frontal and temporal lobes and then gradually spreading to other parts of the brain. A major pathological feature of Alzheimer's disease is the formation of neurofibrillary tangles (NFTs) in neurons by the amyloid plaques due to accumulation of amyloid beta protein and the tau protein involved in microtubules. The formation of amyloid plaques and neurofibrillary tangles (NFTs) leads to neurodegeneration, synaptic dysfunction and dementia.

Amyloid plaques are produced by amyloid precursor protein (APP). Amyloid precursor protein is a transmembrane protein that penetrates the cell membrane of neurons and is very important for neuron growth, survival and repair after damage. When Alzheimer's disease develops, amyloid precursor protein (APP) is digested and cleaved by γ-secretase and β-secretase to produce amyloid beta composed of 39˜43 amino acids. This amyloid beta forms amyloid plaques, a densely accumulated mass, on the outside of neurons.

Although a fundamental treatment method for Alzheimer's disease has not been developed so far, acetylcholinesterase inhibitors are being used as therapeutic agents in each country. However, the drug cannot completely stop the progression of the disease, and only has the effect of alleviating some pathological symptoms or slowing the progression. Drugs in this class include donepezil, rivastingmine, galantamine, and tacrine.

The nervous system consists of neurons and neuroglial cells. Neuroglial cells are cells that support the nervous system and constitute about 90% of the nervous system, supply substances necessary for neurons, and maintain homeostasis for a suitable chemical environment. Unlike neurons that transmit information, neuroglial cells do not have the ability to transmit information, and unlike neurons, they can be recovered after damage. Therefore, cancer that occurs in the brain occurs in glial cells, not neurons. Glial cells are the most distributed cells in the brain. The size of glial cells is about a tenth of that of nerve cells, but it is about ten times the number, and it is estimated that there will be hundreds of billions of cells. Glial cells present in the central nervous system include astrocytes that maintain blood-brain barrier, absorb glucose from blood and supply it to neurons, and help tissue regeneration to improve microenvironment; oligodendrocytes that form myelin sheath of the central nervous system; and microglia and radial glia that serve as immune cells of the central nervous system. Glial cells present in the peripheral nervous system include Schwann cells that form myelin sheath of the peripheral nervous system and satellite cells that supply nutrients to neurons.

Stem cells are considered as a promising treatment for intractable neurological diseases such as stroke because of their ability to replace damaged or lost cells in the nervous system. It has been reported that embryonic stem cells (ESCs) and induced pluripotent stem cells (iPSCs) can differentiate into various neurons and replace the damaged nervous system. However, clinical trials have not been made because of the problem that these cells can cause unwanted cancer. Therefore, ongoing clinical trials are using adult stem cells, particularly early secreted human mesenchymal stem cells (hMSCs).

Mesenchymal stem cells (MSCs) are capable of self-renewal while maintaining growth in the laboratory, and can be differentiated into various types of cells (Bianco et al., 2001; Pittenger et al., 1999; Prockop, 1997). In addition, MSCs have the potential to trans-differentiate into various neuron-like cells having neuronal activity (Munoz-Elias et al., 2003; Sanchez-Ramos et al., 2000; Trzaska et al., 2007; Woodbury et al., 2000). Human mesenchymal stem cells (hMSCs) are being studied as a cell therapy product to treat the damaged nervous system due to their high plasticity and low immune rejection properties (Thuret et al., 2006). The present inventors demonstrated that transplanted hMSCs increase the growth of damaged nerve fibers and the survival of spinal cord cells in an ex vivo spinal cord injury model (Cho et al., 2009).

Mesenchymal stem cells of adult stem cells include early-passage hMSCs and late-passage hMSCs (10 passages or more). Late-passage hMSCs secrete less growth factors or cytokines, resulting in decreased paracrine effect and decreased nervous system recovery. Therefore, early-passage hMSCs are used for the treatment of the damaged nervous system, but in the initial stage of culture, the amount of hMSCs that can be obtained is very limited, so it is difficult to obtain a sufficient amount for treatment.

Thus, the present inventors induced differentiation of mesenchymal stem cells (hMSCs) obtained from human adult stem cells to have the characteristics of neuroglial cells in order to solve the above problems. The present inventors confirmed that the mesenchymal stem cells induced into glia-like cells (glia-like cells induced by hMSCs, hereinafter abbreviated as ghMSCs) were differentiated into glia-like cells that secrete a large amount of growth factors and cytokines, thereby enhancing the paracrine effect and thus exhibiting an excellent effect in Alzheimer's treatment.

Accordingly, the present inventors induced differentiation of mesenchymal stem cells to have the characteristics of glial cells. When the differentiated glia-like cells were co-cultured with neural stem cells having toxicity induced by amyloid beta, effects of increasing the reduced viability and proliferative potential of the neural stem cells and reducing the increased cytotoxicity of the neural stem cells were verified. In addition, the expression of inflammasomes was reduced, and effects of improving long-term memory with respect to spatial perception ability and enhancing spatial cognitive ability in Alzheimer-induced mouse models were verified. The present inventors completed the present invention by confirming the above results.

### SUMMARY OF THE INVENTION

It is an object of the present invention to provide a pharmaceutical composition comprising late-passage human mesenchymal stem cells induced into glia-like cells as an active ingredient for Alzheimer's treatment.

It is another object of the present invention to provide a method for treating Alzheimer's disease comprising a step of administering late-passage human mesenchymal stem cells induced into glia-like cells (ghMSCs) to a subject.

To achieve the above objects, the present invention provides a pharmaceutical composition comprising late-passage human mesenchymal stem cells induced into glia-like cells as an active ingredient for Alzheimer's treatment.

In addition, the present invention provides a method for treating Alzheimer's disease comprising a step of administering late-passage human mesenchymal stem cells induced into glia-like cells (ghMSCs) to a subject.

### Advantageous Effect

The present invention relates to a pharmaceutical composition comprising late-stage human mesenchymal stem cells (ghMSCs) induced into glia-like cells as an active ingredient for Alzheimer's treatment. When the glia-like cells differentiated from the late-stage human mesenchymal stem cells were co-cultured with neural stem cells having toxicity induced by amyloid beta, effects of increasing the reduced viability and proliferative potential of the neural stem cells and reducing the increased cytotoxicity of the neural stem cells were verified. In addition, the expression of inflammasomes is reduced, and effects of improving long-term memory with respect to spatial perception ability and enhancing spatial cognitive ability in Alzheimer-induced mouse models were verified. Therefore, the pharmaceutical composition of the present invention can be advantageously used in Alzheimer's treatment.

### BRIEF DESCRIPTION OF THE DRAWINGS

FIG. 1a is a set of photographs showing the morphological changes of glia-like cells in the ghMSC induction process confirmed by bright-field images.

FIG. 1b is a set of graphs showing the expression levels of the astrocyte markers GFAP, S100β, SLC1A3 and SLC1A2; the oligodendrocyte marker Sox10; the oligodendrocyte progenitor cell marker PDGFRα; and PAPP-A, the marker that plays an important role in ischemic conditions, in human mesenchymal stem cells (hMSCs) and glia-like cells derived from hMSCs (ghMSCs).

FIG. 1c is a set of photographs showing the expressions of the astrocyte markers GFAP and S100β, confirmed by immunohistochemical staining.

FIG. 1d is a diagram showing the expression ratio of the astrocyte markers GFAP and S100β in all glia-like cells, respectively.

FIG. 2 is a graph showing that the viability of neural stem cells was decreased by amyloid beta, but the viability of neural stem cells was increased when amyloid beta and insert well containing glia-like cells or conditioned medium (CM) of ghMSCs were treated together, confirmed by CCK-8 assay.

FIG. 3 is a graph showing that the viability of neural stem cells was decreased by amyloid beta, but the viability of neural stem cells was increased when amyloid beta and glia-like cells insert well or glial cell conditioned medium (CM) of ghMSCs were treated together, confirmed by trypan blue staining.

FIG. 4 is a graph showing that the cytotoxicity of neural stem cells increased by amyloid beta toxicity was reduced due to co-culture with glia-like cells, confirmed by lactate dehydrogenase (LDH) level measurement.

FIG. 5 is a graph showing that the proliferative capacity of neural stem cells reduced by amyloid beta toxicity was increased by co-culture with glia-like cells, confirmed by BrdU assay.

FIG. 6a is a diagram showing the expression changes of the inflammasomes NLRP3 and caspase-1; and the pro-inflammatory cytokine IL-1β in neural stem cells treated with amyloid beta (NSC+Aβ) and glia-like cells (NSC+Aβ+ghMSC).

FIG. 6b is a graph showing the expression of the inflammasome NLRP3 in neural stem cells (NSC), neural stem cells treated with amyloid beta (NSC+Aβ) and glia-like cells (NSC+Aβ+ghMSC), which confirming that the expression of NLRP3 increased by amyloid beta was significantly decreased by the treatment of glia-like cells.

FIG. 6c is a graph showing the expression of the inflammasome caspase-1 in neural stem cells (NSC), neural stem cells treated with amyloid beta (NSC+Aβ) and glia-like cells (NSC+Aβ+ghMSC), which confirming that the expression of caspase-1 increased by amyloid beta was significantly decreased by the treatment of glia-like cells.

FIG. 6d is a graph showing the expression of the pro-inflammatory cytokine IL-1β in neural stem cells (NSC), neural stem cells treated with amyloid beta (NSC+Aβ) and glia-like cells (NSC+Aβ+ghMSC), which confirming that the expression of IL-1β increased by amyloid beta was significantly decreased by the treatment of glia-like cells.

FIG. 7 is a graph showing the escape latency in the vehicle administration group, the human mesenchymal stem cell (hMSC) administration group, and the glia-like cell (ghMSC) administration group, which confirming that glia-like cells (ghMSC) showed the effects of improving long-term memory with respect to spatial perception ability.

FIG. 8 is a graph showing the escape latency in the vehicle administration group, the human mesenchymal stem cell (hMSC) administration group, and the glia-like cell (ghMSC) administration group, which confirming that glia-like cells (ghMSC) showed the effects of improving memory and spatial cognitive ability decline due to Alzheimer's disease.

### DESCRIPTION OF THE PREFERRED EMBODIMENTS

The present invention provides a pharmaceutical composition comprising late-passage human mesenchymal stem cells induced into glia-like cells (ghMSCs) as an active ingredient for Alzheimer's treatment.

In addition, the present invention provides a method for treating Alzheimer's disease comprising a step of administering late-passage human mesenchymal stem cells induced into glia-like cells (ghMSCs) to a subject.

There is a report that the use of neurons or glial cells differentiated from hMSCs can be used as a neuroconstructive approach for neurological disorders. In particular, the paracrine activity of hMSCs is considered to have a significant impact on clinical trials. However, most clinical trials have only used early-passage hMSCs lower than passage 5, despite the high cost, because the paracrine activity of late-passage cells is remarkably low. That is, as confirmed by the present inventors, transplantation of late-passage hMSCs from passages 11 to 14 had little effect on the behavioral recovery of Alzheimer's disease-induced mice.

The present inventors obtained ghMSCs with increased paracrine activity by inducing differentiation of late-passage hMSCs with low neuronal function recovery effect into glia-like cells. In a cell model of Alzheimer's in which neural stem cells were treated with amyloid beta, it was confirmed that the cell viability and proliferation were increased and the cytotoxicity was decreased when glia-like cells were treated. In in vivo experiments, the neurobehavioral function of the mouse model was remarkably restored by ghMSC transplantation. The above results indicate that the ghMSCs derived from late-passage hMSCs are a desirable cell source for treating Alzheimer's disease.

The glia-like cells are at least one selected from the group consisting of oligodendrocytes, astrocytes, microglia, and radial glia.

The late-passage human mesenchymal stem cells are the cells have been passaged 10 to 15 times.

Frozen vials were prepared by putting the glia-like cells differentiated from human mesenchymal stem cells, FBS stock solution and DMSO in DMEM containing fetal bovine serum (FBS), and the prepared frozen vials were placed in a freezing container containing isopropanol. After overnight storage in a −80° C. DEEP freezer, the prepared frozen vials were placed in a liquid nitrogen tank (LN2 tank) and frozen.

The frozen glia-like cells differentiated from human mesenchymal stem cells were thawed in a 37° C. water bath, and the cells in a frozen vial with a little ice were mixed well using a pipette in a biosafety hood. The cells were mixed with DMEM containing fetal bovine serum (FBS) and placed in a conical tube, and the cell pellet precipitated by centrifugation was mixed with DMEM containing fetal bovine serum (FBS). The cells were mixed with trypan blue and smeared on a cell culture dish for use.

The effective dose of the composition is 10³˜10⁹ cells/kg, preferably 10⁴˜10⁸ cells/kg, and more preferably 6×10⁵˜6×10⁷ cells/kg cells/kg. The composition may be administered 2˜3 times a day. The above conditions are not necessarily limited thereto, and may vary depending on the patient's condition and the degree of onset of disease.

The glia-like cells increase the cell viability of neural stem cells, which was reduced by amyloid beta.

The glia-like cells increase the cell proliferation capacity of neural stem cells, which was reduced by amyloid beta.

The glia-like cells reduce the cytotoxicity of neural stem cells, which was increased by amyloid beta.

The composition increases the viability of neural stem cells.

The composition reduces the expressions of inflammasome factors.

The inflammasome is at least one selected from the group consisting of NLRP3 (NLR family pyrin domain-containing protein 3), caspase-1, and IL-1β.

The composition enhances the hippocampus-dependent spatial learning ability.

The composition improves the spatial cognitive ability.

The pharmaceutical composition according to the present invention can contain 10˜95 weight % of glia-like cells differentiate from human mesenchymal stem cells based on the total weight of the composition. In addition, the pharmaceutical composition of the present invention can further include at least one active ingredient exhibiting the same or similar function in addition to the active ingredient.

The glia-like cells differentiated from human mesenchymal stem cells of the present invention can be present as a pharmaceutical composition for treatment. Such a pharmaceutical composition can include a physiologically acceptable matrix or physiologically acceptable excipient in addition to the cells. The type of matrix and/or excipient depends on others according to the intended route of administration. The pharmaceutical composition can also optionally contain other suitable excipients or active ingredients used together in the treatment with stem cells

In addition, the dosage of the composition can be increased or decreased depending on the route of administration, the degree of disease, the patient's gender, body weight, and age, etc. Therefore, the above dosage does not limit the scope of the present invention in any way.

The term “administration” in the present invention means introducing a predetermined substance into a patient by an appropriate method, and the composition can be administered through any general route as long as it can reach a target tissue. The route can be intraperitoneal administration, intravenous administration, intramuscular administration, subcutaneous administration, intradermal administration, oral administration, topical administration, intranasal administration, intrapulmonary administration, or rectal administration, but not always limited thereto.

The composition can be formulated as injectable solutions for parenteral administration such as sterilized solutions or suspensions by mixing with water or other pharmaceutically acceptable liquids. Particularly, the composition can be mixed with pharmaceutically acceptable carriers or mediums, such as sterilized water, saline, vegetable oil, emulsifiers, suspensions, surfactants, stabilizers, excipients, vehicles, antiseptics and binders, and then formulated as a unit dosage authorized by manufacture of medicines. The effective dose of the composition of the present invention means the dose inducing proper amount within designated conditions. Sterile composition for the injection can be provided by using an excipient such as distilled water for injection. To prepare injectable solutions, isotonic solution including saline, glucose or other adjuvants, which is exemplified by D-sorbitol, D-mannose, D-mannitol, and sodium chloride can be co-used with alcohol, particularly ethanol, polyalcohol such as propylene glycol, polyethylene glycol and non-ionic surfactant polysorbate 80™, HCO-50. The oil is exemplified by sesame oil and soybean oil, which can be used together with such solubilizing agents as benzyl benzoate and benzyl alcohol. The oil can also be mixed with buffers such as phosphate buffer, sodium acetate buffer; soothing agents such as procaine hydrochloride; stabilizers such as benzyl alcohol and phenol; and antioxidants. The prepared injectable solution is filled in proper ampoules.

The preferable administration method to a patient is parenteral administration. Specifically, one-time administration to the injured site is basic, but multiple administrations are also acceptable. Administration time can be short or long. Examples of formulations for preferred administration are injections and percutaneously administrable preparations.

In addition, the pharmaceutical composition of the present invention can be administered by any device capable of transporting an active substance to target cells. Preferred administration ways and formulations are intravenous injections, subcutaneous injections, intradermal injections, intramuscular injections, drip injections, and the like. Injections can be prepared using aqueous solvents such as physiological saline and Ringer's solution, vegetable oil, higher fatty acid esters (eg, ethyl oleate), and non-aqueous solvents such as alcohols (eg, ethanol, benzyl alcohol, propylene glycol, glycerin, etc.), and can include pharmaceutical carriers such as stabilizers for preventing deterioration (eg, ascorbic acid, sodium hydrogen sulfite, sodium pyrosulfite, BHA, tocopherol, EDTA, etc.), emulsifiers, buffers for pH adjustment, and preservatives for inhibiting the growth of microorganisms (eg, phenylmercuric nitrate, thimerosal, benzalkonium chloride, phenol, cresol, benzyl alcohol, etc.).

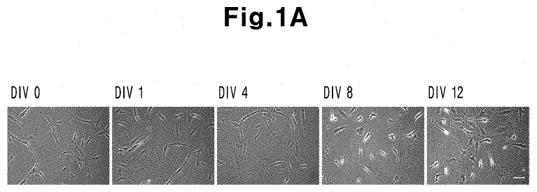

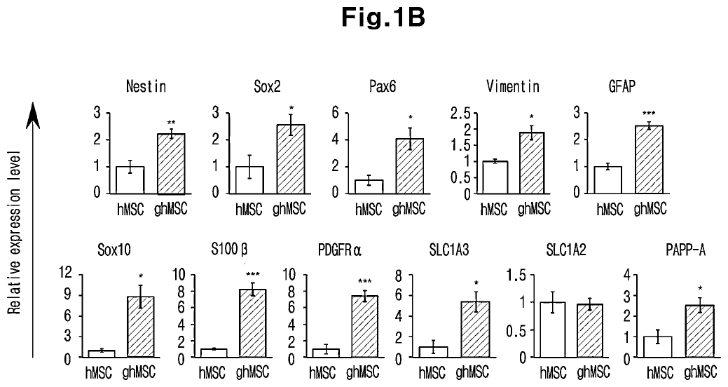

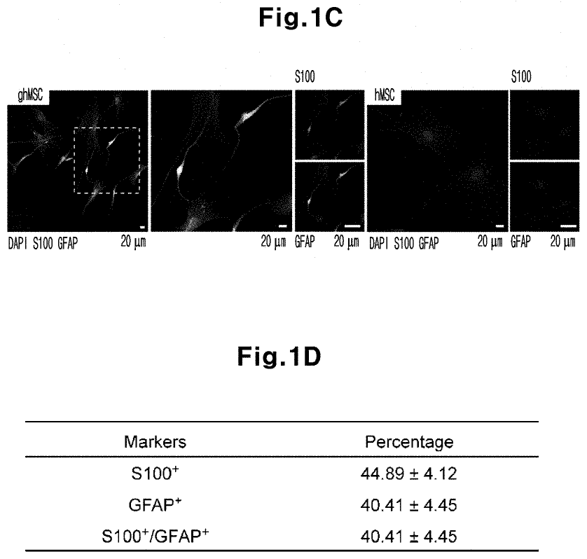

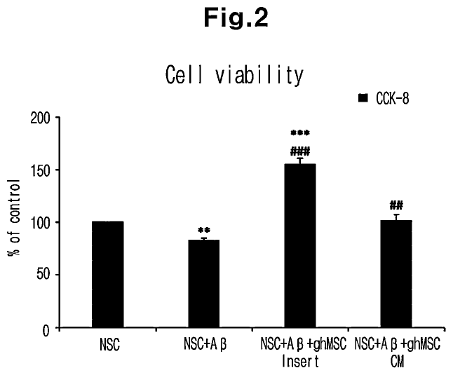

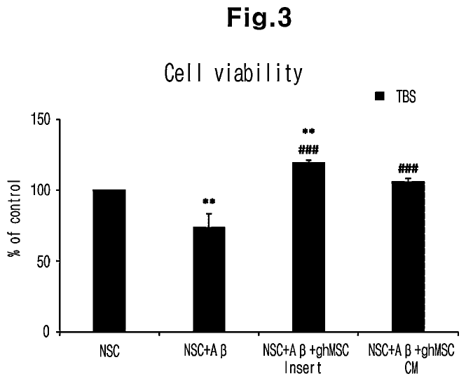

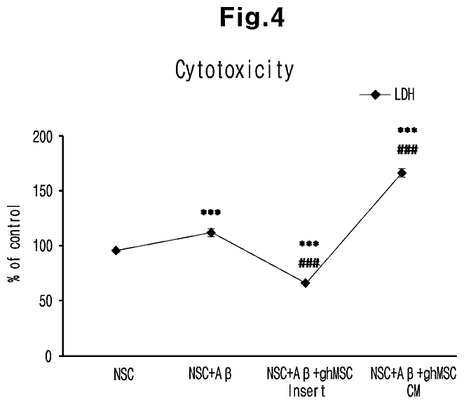

In a specific embodiment of the present invention, glia-like cells were induced from human mesenchymal stem cells (FIGS. 1a˜1d), and in order to confirm the therapeutic effect of the glia-like cells on Alzheimer's disease, neural stem cells were treated with the glia-like cells. As a result, the viability of neural stem cells was increased (see FIGS. 2 and 3), and the cytotoxicity of neural stem cells, which was increased by amyloid beta toxicity, was reduced by co-culture with glia-like cells (see FIG. 4).

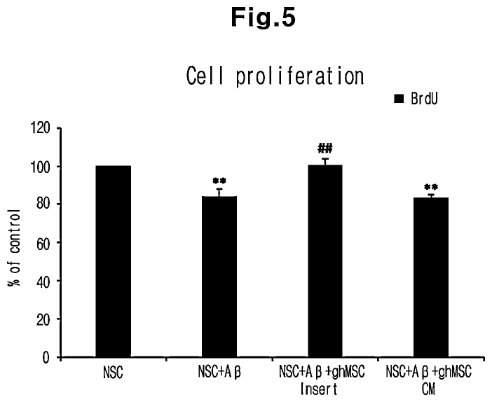

In addition, it was confirmed that the proliferative capacity of neural stem cells, which was reduced by amyloid beta toxicity, was increased by co-culture with glia-like cells (see FIG. 5).

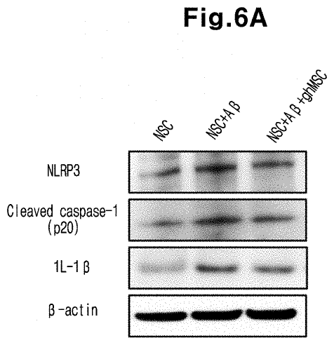

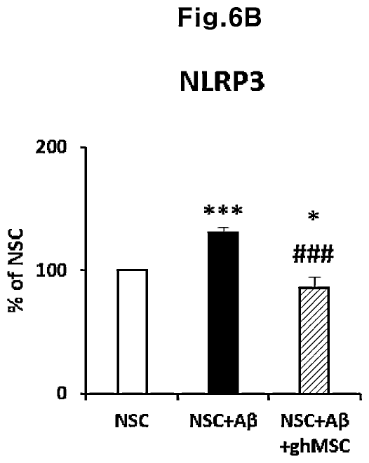

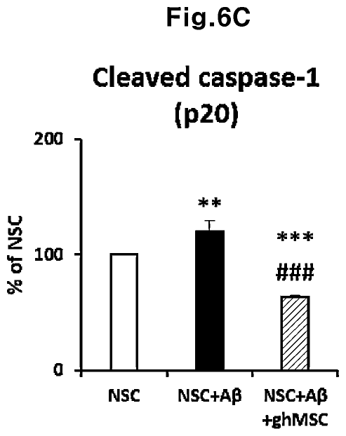

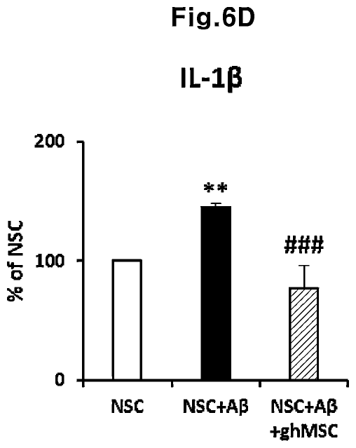

It was also confirmed that the expressions of the inflammasomes NLRP3 and caspase-1; and the proinflammatory cytokine IL-1β, which were increased due to increased inflammatory response, in neural stem cells treated with amyloid beta for 2 hours were significantly decreased by co-culture with glia-like cells (see FIGS. 6a˜6d).

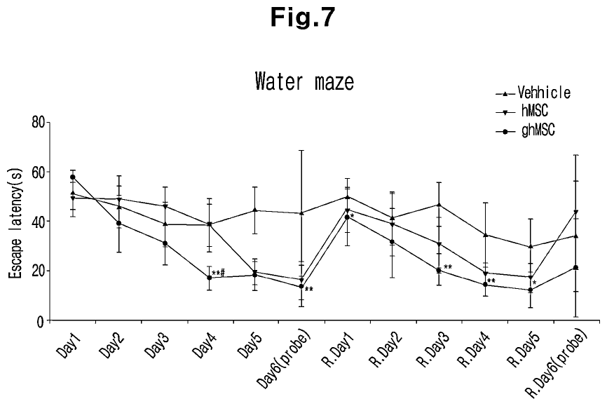

The escape latency was reduced in the group treated with human glia-like cells (ghMSC) compared to the control group treated with human mesenchymal stem cells (hMSC), confirming that human glia-like cells (ghMSC) showed the effects of improving long-term memory with respect to spatial perception ability (see FIG. 7).

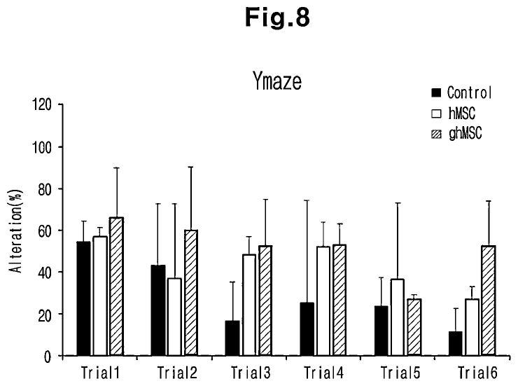

In addition, the memory and spatial cognitive ability were improved in the group treated with ghMSC compared to the control group, confirming that human glia-like cells (ghMSC) showed the effects of improving memory and spatial cognitive ability decline due to Alzheimer's disease (see FIG. 8).

Therefore, the pharmaceutical composition of the present invention can be effectively used for the treatment of Alzheimer's disease.

Hereinafter, the present invention will be described in detail by the following examples and experimental examples.

However, the following examples and experimental examples are only for illustrating the present invention, and the contents of the present invention are not limited thereto.

### Example 1: Culture of Neural Stem Cells

All the animal-related procedures were performed in accordance with Hanyang University guidelines for the management and use of laboratory animals, and approved by the Hanyang University Institutional Animal Care and Use Committee (IACUC). Every effort was made to minimize the number of animals used and their suffering, and all animals were used only once.

Embryonic neural stem cells were obtained from the frontal lobe of rats at 13 to 14 weeks of gestation, transferred to a 100 mm Petri dish containing cold Hank's balanced salt solution (HBSS; 137 mM NaCl, 5.4 mM KCl, 0.3 mM Na₂HPO₄, 0.4 mM KH₂PO₄, 5.6 mM glucose, and 2.5 mM HEPES) (Gibco, BRL, NY, USA), and washed several times with the same solution.

Neural stem cells were isolated from the frontal lobe, lateral ganglionic eminence, and ventral midbrain of rat embryos, and spread on a culture dish pre-coated with poly-L-ornithine/fibronectin dissolved in Ca²⁺/Mg²⁺-free PBS (PBS). The cells were cultured in N2 medium (DMEM/F12, 25 mg/L of insulin, 100 mg/L of transferrin, 30 nM selenite, 100 μM putrescine, 20 nM progesterone, 0.2 mM ascorbic acid, 2 nM L-glutamine, 8.6 mM D(+)glucose, 20 nM NaHCO₂, Sigma, St. Louis, Mo., USA) containing basic fibroblast growth factor (bFGF; 10 ng/ml, Gibco, Frederick, Md., USA). The culture medium was replaced every 2 to 3 days, and the culture was maintained for 4 to 6 days while maintaining the temperature of 37° C. and 5% CO₂ environment to primary culture of embryonic neural stem cells.

### Example 2: Culture of Human Mesenchymal Stem Cells (hMSCs)

Adult human mesenchymal stem cells (hMSCs, Cambrex Bioscience, Walkersville, Md., USA) extracted from normal human bone marrow were cultured in low-glucose Dulbecco's modified Eagle's medium (DMEM) supplemented with 10% fetal bovine serum (FBS; Gibco, Waltham, Mass., USA). Cells (passages 6-12) were passaged 12 to 15 times in an environment of 37° C. and 5% CO₂ to obtain late-passage hMSCs.

### Example 3: Induction of Human Mesenchymal Stem Cells (hMSCs) into Glia-Like Cells (ghMSCs)

The late-passage human mesenchymal stem cells obtained in Example 2 were cultured for 24 hours in the primary differentiation medium (DMEM-low-glucose medium, 10% FBS, 1% penicillin-streptomycin mixture, 1 mM β-mercaptoethanol). Then, the human mesenchymal stem cells were washed with PBS (phosphate buffered solution) and cultured in the secondary differentiation medium [DMEM-low-glucose medium, 10% FBS, 1% penicillin-streptomycin mixture, 0.28 μg/ [symbol] of tretinoin (all-trans-retinoic acid)]. After 3 days, the mesenchymal stem cells were washed with PBS and then cultured in the tertiary differentiation medium (DMEM-low-glucose medium, 10% FBS, 1% penicillin-streptomycin mixture, 10 μM forskolin, 10 ng/ml of human basic-fibroblast growth factor, 5 ng/ml of human platelet derived growth factor-AA, 200 ng/ml of heregulin-β1) for 8 days. At this time, the tertiary differentiation medium was replaced once every two days. The morphology of the hMSCs in culture for the induction of differentiation was observed under a microscope. Cell survival during the induction of differentiation was observed through bright-field imaging (FIG. 1a).

### Example 4: Preparation of Conditioned Medium of ghMSCs (ghMSC-CM)

Glia-like cells (ghMSCs, 1×10⁴ cells/cm²) were rinsed twice with PBS and cultured in serum-free Neurobasal-A medium (NB medium) for 18 hours. The medium was collected and centrifuged at 1500 g for 5 minutes to remove cell debris, and then used for the experiments.

### Example 5: Freezing and Thawing

### <5-1> Freezing of Glia-Like Cells

A frozen vial was prepared by adjusting the total volume to 1 [symbol] by mixing 1˜2×10⁵ human mesenchymal stem cells (or glia-like cells) in DMEM containing 10% fetal bovine serum (FBS), FBS stock solution, and DMSO in a ratio of 5:4:1, respectively. The prepared frozen vial was placed in a freezing container containing 100% isopropanol. The prepared vial was stored overnight in a −80° C. DEEP freezer, and then stored in a liquid nitrogen tank (LN2 tank).

### <5-2> Thawing of Glia-Like Cells

The frozen vial stored in the liquid nitrogen tank was thawed in a 37° C. water bath for about 2 minutes, and the cells in the frozen vial with a little ice were mixed well using a pipette in a biosafety hood. 1 [symbol] of the cell concentrate in the frozen vial was mixed with 10 [symbol] of DMEM containing 10% FBS and placed in a 15 [symbol] conical tube. The cell pellet was precipitated by centrifugation at 1,200 rpm for 5 minutes, followed by sucking all of the supernatant. 1 [symbol] of DMEM containing 10% fetal bovine serum (FBS) was placed in the 15 [symbol] conical tube, and the cell pellet was suspended using a pipette. 11 [symbol] of the cell pellet was mixed with 11 [symbol] of trypan blue. 10 [symbol] of the mixture was injected into a cell counter to calculate the number of cells contained in 1 [symbol] , and plated on a cell culture dish at the density of 2,000 cells/cm².

### Experimental Example 1: Characterization of Glia-Like Cells (ghMSCs) Derived from Late-Passage Human Mesenchymal Stem Cells

Glia-like cells were induced from human mesenchymal stem cells by the method of Example 3. Inducing glia-like cells and induced glia-like cells were frozen and thawed, and then used for analysis. To confirm the characteristics of the induced glia-like cells, the expression levels of glia markers were confirmed by quantitative RT-PCR and immunohistochemical staining.

Particularly, for quantitative RT-PCR, RNA was extracted using TRIzol reagent (Invitrogen, Carlsbad, Calif., USA) and treated with DNase. For cDNA synthesis, reverse transcription was performed at 42° C. for 1 hour using M-MLV reverse transcriptase (Promega). SYBR FAST qPCR Kits (KAPA Biosystems) were used to identify the genes expressed in cDNA with the respective gene-specific primers represented by SEQ. ID. NO: 1˜NO: 24. The expression level of the target gene was confirmed based on GAPDH using a Ct method. The ΔCt value is the value minus the Ct value, and the cycle number of the critical value was measured as follows: ΔCt=Ct(Target)−Ct(GAPDH). The relative value of the target gene to endogenous GAPDH was determined by fold-change of GAPDH=2^{-ΔCt}.

In addition, for immunohistochemical staining, the cultured cells were fixed with 4% paraformaldehyde (PFA), and cultured with a blocking solution containing 5% standard goat serum and 0.1% Triton-X100. The cells were stained with GFAP (1:200; Merck Millipore) and 5100 (1:250; Dako) for one day in a 4° C. refrigerator. The staining solution was washed with PBS several times, and the cells were stained with the secondary antibodies Alexa Fluor® 488 anti-mouse IgG (Molecular Probes) and Alexa Fluor® 546 anti-rabbit IgG (Molecular Probes) for 1 hour at room temperature. Then, the nuclei were stained with 4,6-diamidino-2-phenylindole (DAPI) (Santa Cruz Biotechnology). The samples were observed with a digital inverted fluorescence microscope (DM5000B; Leica).

As a result, as shown in FIG. 1b, it was confirmed that the expression levels of the radial glia markers Nestin, Sox2, Pax6, Vimentin and GFAP; the astrocyte markers GFAP, S100β and SLC1A3; and the oligodendrocyte marker Sox10 were increased in ghMSCs than in hMSCs. It was also confirmed that the expression levels of IGFBP-4 and IGF-1 pregnancy-associated plasma protein-A (PAPP-A) were increased in ghMSCs than in hMSCs (FIG. 1b). In addition, as shown in FIGS. 1c and 1d, it was confirmed that the oligodendrocyte markers Sox10 and GFAP were expressed in 45% and 40% of total glia-like cells (ghMSCs), respectively. The above results suggest that the glia-like cells derived from human mesenchymal stem cells contain a lot of astrocytes and oligodendrocytes that help the growth of surrounding cells and improve the surrounding environment, so these cells have the potential to treat stroke.

### Experimental Example 2: Confirmation of Effect of Glia-Like Cells (ghMSCs) Differentiated from Human Mesenchymal Stem Cells on Survival and Proliferation of Damaged Neural Stem Cells

<2-1> Preparation of Amyloid Beta Peptide Oligomer and Conditioned Media (CM) of Glia-Like Cells (ghMSCs) Differentiated from Human Mesenchymal Stem Cells

Amyloid beta peptide (Sigma) was first dissolved in dimethyl sulfoxide (DMSO; Panreac, Barcelona, Spain) at the concentration of 5 mM, and then DMEM/F-12 medium (Gibco) was added thereto to make the final concentration of 1 mM, and the amyloid beta peptide oligomer was prepared by incubating at 4° C. for 24 hours.

<2-2> Measurement of Viability of Neural Stem Cells (NSCs) According to Treatment of Glia-Like Cells (ghMSCs) Differentiated from Human Mesenchymal Stem Cells (1)

Cell viability of neural stem cells, in which toxicity was induced by amyloid beta, was measured when co-cultured with glia-like cells (ghMSCs) by CCK-8 assay.

Particularly, the CCK-8 assay is an analytical method used to measure the viability of neural stem cells by confirming the degree of binding between WST-[2-(2-methoxy-4-nitrophenyl)-3-(4-nitrophenyl)-5-(2,4-disulfophenyl)-2H-tetrazolium, monosodium salt] and 1-methoxy PMS. To confirm the effect of glia-like cells on the neurotoxicity induced by amyloid beta oligomer, neural stem cells were seeded in the pre-coated 24-well plate at the density of 1×10⁵ cells/cm². The 20 μM amyloid beta oligomer prepared in Experimental Example 2-1 and the 0.4 μm pore insert well in which glia-like cells were seeded at the density of 8×10³ cells/cm² were treated thereto. The well plate coated with neural stem cells was simultaneously treated with the amyloid beta oligomer and the ghMSC conditioned medium (ghMSC-CM) prepared in Example 4 for 48 hours. Then, neural stem cells were treated with 300 [symbol] of culture medium and 30 [symbol] of CCK-8 reagent, followed by culture for 2 hours. The viability of neural stem cells was measured at 450 nm and 650 nm using an ELISA plate reader (Synergy H1 Hybrid reader, BioTek, Winooski, Vt., USA).

As a result, although the viability of neural stem cells was decreased by amyloid beta (NSC+Aβ), the viability of neural stem cells was increased in the groups treated with amyloid beta and glia-like cells (NSC+Aβ+ghMSC insert) and the conditioned medium of glia-like cells were treated together (NSC+Aβ+ghMSC CM) (FIG. 2).

<2-3> Measurement of Viability of Neural Stem Cells (NSCs) According to Treatment of Glia-Like Cells (ghMSCs) Differentiated from Human Mesenchymal Stem Cells (2)

Cell viability of neural stem cells, in which toxicity was induced by amyloid beta, was measured when co-cultured with glia-like cells (ghMSCs) by trypan blue staining.

Particularly, neural stem cells were treated with amyloid beta oligomer and glia-like cell insert well or the conditioned medium of ghMSCs (ghMSC-CM) at the same time for 48 hours in the same manner and conditions as described in Experimental Example 2-3. Then, neural stem cells were stained with trypan blue solution (Gibco) for 2 minutes, and live and dead cells were counted using a hemocytometer.

As a result, although the viability of neural stem cells was decreased by amyloid beta (NSC+Aβ), the viability of neural stem cells was increased in the groups treated with amyloid beta and glia-like cells (NSC+Aβ+ghMSC insert) and the conditioned medium of glia-like cells were treated together (NSC+Aβ+ghMSC CM) (FIG. 3).

<2-4> Measurement of Cytotoxicity of Neural Stem Cells (NSCs) According to Treatment of Glia-Like Cells (ghMSCs) Differentiated from Human Mesenchymal Stem Cells

It was confirmed through lactate dehydrogenase (LDH) analysis that the toxicity of neural stem cells induced by amyloid beta was reduced by co-culture with glia-like cells.

Particularly, a colorimetric assay kit (Roche Boehringer-Mannheim, Indianapolis, Ind., USA) is used to quantify the cytotoxicity of neural stem cells releasing lactate dehydrogenase (LDH). Neural stem cells were treated with amyloid beta oligomer and glia-like cell insert well or the conditioned medium of ghMSCs (ghMSC-CM) at the same time for 48 hours in the same manner and conditions as described in Experimental Example 2-3. Neural stem cells were centrifuged at 200 g for 10 minutes at 27° C. Then, the supernatant was transferred to a new 96-well plate according to the manufacturer's instructions, to which colorimetric solution was added. The plate was incubated for 30 minutes while blocking light. The cytotoxicity was measured at 492 nm and 690 nm with an ELISA reader.

As a result, it was confirmed that the cytotoxicity of neural stem cells, which was increased by amyloid beta toxicity, was reduced by co-culture with glia-like cells (FIG. 4).

<2-5> Measurement of Proliferative Capacity of Neural Stem Cells (NSCs) According to Treatment of Glia-Like Cells (ghMSCs) Differentiated from Human Mesenchymal Stem Cells

To confirm the effect of amyloid beta and glia-like cells on the proliferative capacity of neural stem cells, BrdU assay was performed.

Particularly, neural stem cells were treated with amyloid beta oligomer and glia-like cell insert well or the conditioned medium of ghMSCs (ghMSC-CM) at the same time for 48 hours in the same manner and conditions as described in Experimental Example 2-3. The cells were labeled with 10 μM BrdU for 18 h using a BrdU labeling and detection kit (Roche Boehringer-Mannheim) according to the manufacturer's instructions and fixed with a fixative solution for 30 minutes. 300 [symbol] of an anti-BrdU-POD working solution was added to the fixed cells and incubated for 2 hours while blocking light. The cells were washed three times with a washing solution and then reacted with 300 [symbol] of a substrate. After culturing the cells for 5 minutes while light blocking, the cell proliferation was measured at 370 nm and 492 nm using an ELISA reader.

As a result, as shown in FIG. 5, It was confirmed that the proliferative capacity of neural stem cells, which was reduced by amyloid beta toxicity, was increased by co-culture with glia-like cells (n=4).

### Experimental Example 3: Measurement of Expression Changes of Inflammasome

To confirm the expression levels of inflammasome factors such as NLRP3, caspase-1 and IL-1β when co-cultured with glia-like cells in neural stem cells, in which toxicity was induced by amyloid beta, Western blotting was performed.

Particularly, neural stem cells were treated with amyloid beta oligomer and glia-like cell-enriched solution for 48 hours in the same manner and conditions as described in Experimental Example 2-3. The cells were collected with a scraper and centrifuged at 6,000×g for 2 minutes at 4° C. The cell pellet was rinsed twice with cold D-PBS, to which lysis buffer (RIPA II cell lysis buffer 1× with Triton, without ethylenediaminetetraacetic acid (EDTA); 1 mM phenylmethylsulfonyl fluoride (PMSF); 1 mM sodium fluoride (NaF); 1 mM sodium orthovanadate (Na₃VO₄); and 0.5% protease inhibitor cocktail 1×) was added, followed by incubation on ice for 30 minutes. Then, the cells were sonicated several times using a sonicator (Sonoplus, Bandelin Electronics, Berlin, Germany) and incubated on ice for another 30 minutes. The cell lysate was centrifuged at 21,100×g at 4° C. for 15 minutes, and the protein concentration was quantified using a bicinchoninic acid (BCA) protein assay kit (Sigma). The lysate samples with equal amounts of protein were loaded on 4-12% sodium dodecyl sulfate-polyacrylamide gel electrophoresis (SDS-PAGE, Invitrogen) gels. After SDS-PAGE, the proteins were transferred to a PVDF membrane (Millipore, Bedford, Mass., USA), and the reaction was blocked with 5% skim milk, followed by incubation with specific primary antibodies. The primary antibodies were: NLRP3 (2 μg/ [symbol] , Novus Biologicals, Littleton, Colo., USA), caspase-1 (2.5 μg/ [symbol] , Novus Biologicals), IL-1β (0.4 μg/ [symbol] , Abcam, Burlingame, Calif., USA), and β-actin (1:4000, Cell signaling, Beverly, Mass., USA). The membrane was rinsed three times with Tris-buffered saline containing 0.1% Tween-20 (TBST) and then incubated with the HRP-conjugated anti-rabbit antibody (1:2000, Jackson ImmunoResearch Laboratories Inc., West Grove, Pa., USA). The membrane was visualized by West-Q Chemiluminescent substrate kit (GenDEPOT, Katy, Tex., USA) and measured with an image analyzer (ImageQuant LAS 4000; GE Healthcare, Little Chalfont, UK).

As a result, as shown in FIGS. 6a˜6d, the inflammatory response was increased in neural stem cells treated with amyloid beta (NSC+Aβ), and the expression levels of the inflammasomes NLRP3 and caspase-1, and the proinflammatory cytokine IL-1β were significantly increased. However, this increased inflammatory response was significantly reduced by co-culture with glia-like cells (NSC+Aβ+ghMSC).

### Experimental Example 4: Confirmation of Therapeutic Effect of Human Glia-Like Cells on Alzheimer's Disease in Alzheimer-Induced Mouse Model

### <4-1> Preparation of Experimental Animals

3×Tg mice (B6; 129, 13 months old, 50±5 g) were purchased from the Jackson laboratory (Bar harbor, ME, USA) and were raised at Hanyang University laboratory animal center (temperature: 22±2° C., relative humidity: 50±10%) in SPF condition with 12/12 hr light/dark cycle (08:00˜20:00). All the mice were allowed to take the irradiated diet freely. All the experiments were carried out after being approved by the Hanyang University Institutional Animal Care and Use Committee (HNU-IACUC) for scientific and ethical evaluation. The experimental animals were divided into 3 groups (vehicle administered 3×Tg group, neural stem cell administered 3×Tg group, and glia-like cell administered 3×Tg group), and 5 animals were assigned to each group. The Alzheimer's disease-induced mouse model was anesthetized using isoflurane (Ifran Liq, Hana Pharm, Korea), and vehicle, neural stem cells or glia-like cells were administered directly into the brain by stereotaxic technique (AP=−2, L=1, V=1.5). The vehicle-administered 3×Tg group was administered with neurobasal media (3 [symbol] ), and the hMSC-administered 3×Tg group and the ghMSC-administered 3×Tg group were each administered intracranially with 2×10⁵ cells of hMSCs and ghMSCs by dispensing in neurobasal media (3 [symbol] ) (once/2 week intervals/4 times). One week after the end of the administration, all the mice were subjected to the behavioral measurements of Experimental Examples 4-2 and 4-3 below.

### <4-2> Confirmation of Effects of Human Glia-Like Cells on Hippocampus-Dependent Spatial Learning Ability in Alzheimer's Disease Mouse Model

In order to confirm the effect of the administration of human glia-like cells on hippocampus-dependent spatial learning ability in a mouse model induced with Alzheimer's disease, Morris water maze test was performed as follows.

Particularly, in the Morris water maze test, learning training was conducted for 5 days and spatial memory was measured on the 6th day. From the 7th day, the test was conducted by changing the location of the escape platform. The Morris water maze test device consists of a circular water pool, an escape platform, and a computerized video-tracking system camera (Jeoungdo B&P, Korea). After filling the pool with water (24±1° C.), about 20 ml of white icing color (Wilton Co, USA) was dissolved in water so that the escape platform was not visible. During the test, the environment in the laboratory, such as the laboratory table, computer, and chair, and the position of the experimenter were also kept constant. The underwater maze was divided into four quadrants: northeast (NE), northwest (NW), southeast (SE), and southwest (SW), and an escape platform was installed in the center of the southwest (SW) quadrant. The water maze learning training was carried out at the same time for 12 days 4 times a day for each experimental animal, and each swim, the animal's head was oriented in different quadrants to start swimming. Experimental animals were allowed to swim freely in the water pool for 60 seconds, finding a hidden escape platform on their own and climbing up. The experimental animals that found the shelter by themselves stayed on the escape platform for seconds and were allowed to observe their surroundings freely, and the time at which they reached the escape platform was recorded.

As shown in FIG. 7, the decrease in escape latency in the Morris water maze indicates long-term memory-related learning ability, and as a result of cognitive training 4 times a day for 12 days, the escape latency was gradually decreased in the human ghMSC administered group compared to the vehicle administered group in which the escape latency did not decrease, showing a significant difference at 3, 4, 6 days and reverse 3, 4, 5 days. On the 4th day, the escape latency was gradually decreased in the group administered with human glia-like cells (ghMSCs) compared to the group administered with human mesenchymal stem cells (hMSCs), a control group, showing a significant difference. These results indicate that human glia-like cells (ghMSCs) have therapeutic effects of improving long-term memory with respect to spatial perception ability.

### <4-3> Confirmation of Effects of Human Glia-Like Cells on Spatial Cognitive Ability in Alzheimer's Disease Mouse Model

In order to confirm the effect of the administration of human glia-like cells on spatial cognitive ability in a mouse model induced with Alzheimer's disease, Y-maze test was performed as follows.

The Y-maze used in this test is made of opaque acrylic material and consists of three arms. After defining each arm as A, B, and C, the experimental animal was placed in the center and the movement path was recorded for 5 minutes (once/2 day intervals/6 times). When entering three different arms in turn, 1 point was given, divided by the total number of passes, and multiplied by 100.

As shown in FIG. 8, as a result of observing the behavior of the experimental animals for 5 minutes, the ghMSC-administered group showed no significant difference compared to the vehicle-administered group, but showed a higher value, resulting in improved memory and spatial cognitive ability. The above results indicate that human glia-like cells (ghMSCs) are effective in improving memory and spatial cognitive ability decline due to Alzheimer's disease.

1. A method for treating Alzheimer's disease in a subject comprising:
administering an effective amount of glia-like cells (ghMSCs) to the subject, wherein the ghMSCs are induced from late-passage human mesenchymal stem cells, thereby treating the Alzheimer's disease in the subject.

2. The method for treating Alzheimer's disease according to claim 1, wherein the ghMSCs are oligodendroglia, astrocytes, microglia, radial glia, or a combination thereof.

3. The method for treating Alzheimer's disease according to claim 1, wherein the late-passage human mesenchymal stem cells have been passaged 10 to 15 times.

4. The method for treating Alzheimer's disease according to claim 1, wherein the ghMSCs are derived from bone marrow, adipose tissue, blood, umbilical cord blood, liver, skin, gastrointestinal tract, placenta or uterus.

5. The method for treating Alzheimer's disease according to claim 1, wherein the composition reduces the expression of an inflammasome factor.

6. The method for treating Alzheimer's disease according to claim 5, wherein the inflammasome factor is NLRP3 (NLR family pyrin domain-containing protein 3), caspase-1, or IL-1β.

7. The method for treating Alzheimer's disease according to claim 1, wherein the method enhances hippocampus-dependent spatial learning ability in the subject.

8. The method for treating Alzheimer's disease according to claim 1, wherein the method improves spatial cognitive ability in the subject.

9. The method for treating Alzheimer's disease according to claim 1, wherein the ghMSCs are administered at a density of 6×10⁵˜6×10⁷ cells/kg.

10. The method for treating Alzheimer's disease according to claim 1, wherein the ghMSCs are administered as a cell therapy product.

11. A method for treating Alzheimer's disease, comprising a step of administering late-passage human mesenchymal stem cells (hMSCs) induced into glia-like cells (ghMSCs) to a subject.
